(19) United States
(12) Patent Application Publication
(10) Pub. No.: US 2025/0341522 A1
(43) Pub. Date: Nov. 6, 2025

(54) Treatment Strategies for Pancreatic Cancer Using Recombinant Acid Sphingomyelinase

(71) Applicant: University of Cincinnati, Cincinnati, OH (US)

(72) Inventors: Gregory C. Wilson, Cincinnati, OH (US); Erich Gulbins, Cincinnati, OH (US); Syed Ahmad, Cincinnati, OH (US)

(73) Assignee: University of Cincinnati, Cincinnati, OH (US)

(21) Appl. No.: 18/653,687

(22) Filed: May 2, 2024

(51) Int. Cl. G01N 33/574; A61K 38/46; A61P 35/00; C12N 9/16

(52) U.S. Cl. CPC G01N 33/57438; A61K 38/465; A61P 35/00; C12N 9/16; C12Y 301/04012

(57) ABSTRACT

A method of treating pancreatic adenocarcinoma in an individual is disclosed. The method involves administering to the individual a therapeutically effective amount of a therapeutic agent comprising recombinant acid sphingomyelinase. In one embodiment, the therapeutic agent further includes one or more compositions selected from the group consisting of modified enzymes, fusion proteins and constitutively active mutants. In another embodiment, the therapeutic agent further includes a pharmaceutically acceptable excipient.

### FIELD OF THE INVENTION

This invention relates generally to treatments for pancreatic cancer.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Pancreatic cancer is on track to become the third leading cause of cancer related death in the world by 2025. Despite decades of advancement and research into the multimodal care of pancreatic adenocarcinoma (PDAC), the outlook remains grim. Five-year overall survival for all patients with pancreatic cancer has only recently risen above 10%. Unfortunately, the majority of patients with PDAC present with metastatic disease at time of diagnosis. Only a minority of patients present with potentially resectable disease and even these patients with “localized disease” experience high rates of distant recurrence and poor overall survival. The current state of PDAC outcomes remains a humbling reminder that significant work remains in our understanding of the molecular basis of this disease and our ability to treat it. Much work is still needed in developing effective treatment strategies to incorporate local and systemic therapies in novel ways to treat the primary tumor as well as micrometastatic disease likely present at the time of diagnosis. It is also necessary to define prognostic factors that allow individual treatments and to characterize signaling mechanisms in the tumor cells and/or host cells that determine tumor progression.

The acid sphingomyelinase (EC 3.1.4.12, sphingomyelin phosphodiesterase 1 (SMPD1); optimal pH 5.0) is a glycoprotein that functions as a lysosomal hydrolase, catalyzing the degradation of sphingomyelin to phosphorylcholine and ceramide. The enzyme mainly localizes to lysosomes, but it is also present in acidic compartments on the cell surface, since lysosomes are constantly recycling to and exchanging with the plasma membrane. Ceramide generated by the acid sphingomyelinase is then further metabolized to sphingosine, which is phosphorylated to sphingosine-1-phosphate. Ceramide can be also phosphorylated to ceramide-1-phosphate or further metabolized to glycosylated sphingolipids. The acid sphingomyelinase, ceramide- and its derivatives have been implied by many studies to be very important in the pathogenesis and treatment of tumors with chemotherapy and irradiation, for the regulation of the immune response and tumor vasculogenesis. It has been shown that the acid sphingomyelinase and ceramide play an important role in receptor signaling and regulation of the innate immune response. The signaling function of the acid sphingomyelinase seems to be mainly regulated by the activity of acid sphingomyelinase on the cell surface resulting in the formation of ceramide in the outer leaflet of the cell membrane. The generation of ceramide molecules within the outer leaflet alters the biophysical properties of the plasma membrane, because the very hydrophobic ceramide molecules spontaneously associate with each other to form small ceramide-enriched membrane domains that fuse and form large, highly hydrophobic, tightly packed, gel-like ceramide-enriched membrane domains. These large, distinct, ceramide-enriched membrane domains have been shown to be crucially involved in cellular stress responses, such as induction of cell death. However, ceramide is also able to bind directly to signaling molecules such as e.g. cathepsins, some PKC isoforms, phosphatase 2A or Lc3B.

Although it is well known that sphingolipids are very important in tumor biology, it is unknown whether expression of the acid sphingomyelinase in malignant tumors, in particular PDAC has an impact on the prognosis and long-term survival of patients.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention involves a method for predicting the survival time of an individual with pancreatic cancer. The method involves the steps of:
a. obtaining a tumor sample from the individual;
b. measuring acid sphingomyelinase expression in the tumor sample;
c. categorizing the level of acid sphingomyelinase expression as high expression or low expression; wherein high expression is determined by a moderate to strong level of acid sphingomyelinase shown in the tumor sample, and low expression is determined by a low to non-detectable level of acid sphingomyelinase shown in the tumor sample; and
d. using the level of acid sphingomyelinase expression to predict survival time of the individual.

In one embodiment, a high level of acid sphingomyelinase expression indicates an increased likelihood of long-term survival and an improved prognosis for the individual. In another embodiment, a low level of acid sphingomyelinase expression indicates a decreased likelihood of long-term survival and a poor prognosis for the individual.

In another embodiment, the present invention involves a method of treating pancreatic adenocarcinoma in an individual in need thereof. The method involves administering to the individual a therapeutically effective amount of a therapeutic agent comprising recombinant acid sphingomyelinase. In one embodiment, the therapeutic agent further includes one or more compositions selected from the group consisting of modified enzymes, fusion proteins and constitutively active mutants. In another embodiment, the therapeutic agent further includes a pharmaceutically acceptable excipient. In one embodiment, the therapeutic agent is administered as one or more doses. In another embodiment, the therapeutic agent is administered parenterally. In one embodiment, the therapeutic agent is administered intravenously. In another embodiment, the therapeutic agent is administered in a continuous infusion.

In another embodiment, the present invention involves a method for predicting the survival time of an individual with pancreatic cancer and treating pancreatic cancer. The method involves the steps of:
e. obtaining a tumor sample from the individual;
f. measuring acid sphingomyelinase expression in the tumor sample;
g. categorizing the level of acid sphingomyelinase expression as high expression or low expression; wherein high expression is determined by a moderate to strong level of acid sphingomyelinase shown in the tumor sample, and low expression is determined by a low to non-detectable level of acid sphingomyelinase shown in the tumor sample;
h. determining a decreased likelihood of long-term survival and a poor prognosis for an individual with a low level of acid sphingomyelinase expression; and
i. administering a therapeutically effective amount of a therapeutic agent comprising recombinant acid sphingomyelinase to an individual with a low level of acid sphingomyelinase expression.

In one embodiment, the therapeutic agent further includes one or more compositions selected from the group consisting of modified enzymes, fusion proteins and constitutively active mutants. In another embodiment, the therapeutic agent further includes a pharmaceutically acceptable excipient. In one embodiment, the therapeutic agent is administered as one or more doses. In another embodiment, the therapeutic agent is administered parenterally. In one embodiment, the therapeutic agent is administered intravenously. In another embodiment, the therapeutic agent is administered in a continuous infusion.

### BRIEF DESCRIPTION OF THE DRAWINGS

The objects and advantages of the disclosed invention will be further appreciated in light of the following detailed descriptions and drawings in which:

FIGS. 1A and 1B demonstrate that acid sphingomyelinase expression correlates with survival in patients with pancreatic ductal adenocarcinoma. FIG. 1A is a series of immunohistochemistry stainings of the acid sphingomyelinase. FIG. 1B is a graph showing overall survival in resectable pancreatic ductal adenocarcinoma.

FIGS. 2A-2D are images showing acid sphingomyelinase expression in human pancreatic ductal adenocarcinoma. Shown are acid sphingomyelinase (SMPD1 is the gene symbol for the acid sphingomyelinase) expression levels in pancreatic adenocarcinoma (compared to normal pancreatic tissue and by cancer stage). FIG. 2A is a graph showing the mRNA expression of SMPDI. FIG. 2B is a violin plot of acid spingomyelinase (SMPD1) expression stratified by pancreatic cancer stage from the TCGA dataset. FIG. 2C is a survival analysis by acid sphingomyelinase expression in the human protein atlas database. FIG. 2D is a graph showing orthotopic PDAC tumor growth in Wildtype or ASM-deficient mice with 106 Pan02 cells. Mean±SD, n=4-5 mice per group; *p<0.05, ANOVA with Tukey post hoc test

FIG. 3 is a pair of graphs showing that functional inhibitors of acid sphingomyelinase during neoadjuvant treatment of PDAC impact prognosis of patients.

FIG. 4 is a graph showing a growth of PDAC in murine models of orthotopic pancreatic ductal adenocarcinoma.

FIG. 5 is a graph showing the survival of wildtype and ASM-transgenic mice after i.p. injection of 25,000 KPC cells. Treatment of ASM-transgenic (tr) mice with gemcitabine rescued 100% of the mice from tumor growth. n=12 each group.

FIG. 6A is a graph showing ASM activity in spleens of wildtype (wt) or ASM-tr mice 14 days after i.p. injection of KPC cells or of vehicle control.

FIG. 6B is a graph showing sphingomyelin levels in spleens of wildtype (wt) or ASM-tr mice 14 days after i.p. injection of KPC cells or of vehicle control.

FIG. 7A is a graph showing the binding of [¹⁴C]sphingomyelin (SM), [¹⁴C]ceramide (Cer), and [¹⁴C]sphingosine (SPH) in the absence or presence of the VMLTLYFYY peptide.

FIG. 7B is a graph showing the results of isolated lysosomes from Ly6G-sorted splenocytes obtained from mice with or without PDAC being loaded with [¹⁴C]arginine and the efflux of [3H]arginine being determined. Aliquots of the lysosomes were treated with sphingomyelin.

FIGS. 8A-8E show the results of phosphorylation of TFEB, Arginase-1- and ADC-expression, and arginine levels that were determined in the spleens of wt and ASM-tr mice with or without PDAC. FIG. 8A is a Western blot with anti-TFEB antibodies. FIG. 8B is a graph showing ELISA for arginase-1. FIG. 8C is a series of images showing confocal microscopy. FIG. 8D is a graph showing ELISA for arginine. FIG. 8E is a graph showing the results of chromium-release assays with splenocytes from wt or ASM-tr mice 14 days after i.p. injection of KPC cells measure cytotoxic T-cell activity. Mean±SD; n=6-8; ***p<0.001 ANOVA, representative blots and microscopy from 2 mice each.

FIG. 9A is a graph showing the concentration of polyamines in the blood plasma of wt or ASM-tr mice determined by ELISA. FIG. 9B is a graph showing the activity of ASM for splenocytes that were isolated from wt or ASM-tr mice and stimulated for 8 hr with 5 μM agmatine (Agm), 100 μM spermine (Sp), or 100 μM spermidine (Spd). FIG. 9C is a graph showing the concentrations of sphingomyelin for splenocytes that were isolated from wt or ASM-tr mice and stimulated for 8 hr with 5 μM agmatine (Agm), 100 μM spermine (Sp), or 100 μM spermidine (Spd). The activity of ASM and the concentrations of sphingomyelin were determined by an enzyme assay or ELISA. Mean±SD; n=6-8; **p<0.01, ***p<0.001 ANOVA.

FIG. 10A is a graph showing ASM activity. FIG. 10B is a graph showing levels of sphingomyelin (B). FIG. 10C is a graph showing levels of Arg-1. FIG. 10D is a graph showing levels of ADC. The levels were determined in macrophages (J774 cell line) or in stable ASM-transfected J774 (with an approximately 3-fold overexpression of ASM) at baseline, after treatment with agmatine (Agm, 5 M), spermine (Sp, 100 μM), or spermidine (Spd, 100 μM), supernatant (Sup) from KPC cells, and supernatant immunodepleted (id) of polyamines. ASM activity was measured by determining the consumption of [¹⁴C]sphingomyelin, Sphingomyelin-, Arginase-1- and ADC-levels were quantified by ELISA. Mean±SD; n=6-8; ***p<0.001, ANOVA

FIG. 11 is a graph showing the results of wt mice that were treated with gemcitabine alone, gemcitabine plus recombinant ASM, or vehicle control after peritoneal injection of KPC cells. Recombinant ASM was administered twice daily at 0.75 mg/kg i.v. for two treatment cycles of 4 days each.

FIG. 12 is a graph showing the PDI expression on lymphocytes of tumor mice upon treatment with recombinant acid sphingomyelinase or solvent.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the agents described herein are administered intravenously.

As used herein the term “fusion protein” means a protein formed by fusing (i.e., joining) all or part of two polypeptides which are not the same.

A “pharmaceutical composition,” as used herein, refers to a composition comprising an active ingredient (e.g., a bacterial cell, an inducer, a drug, or a detectable compound) with other components such as a physiologically suitable carrier and/or excipient.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the patient is a human.

As used herein, a “therapeutic amount,” “therapeutically effective amount,” or “therapeutically effective concentration” of an agent is an amount or concentration of the agent that treats signs or symptoms of a disease (e.g., pancreatic adenocarcinoma) in the subject (e.g., mammal).

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Pancreatic adenocarcinoma (PDAC) is one of the most common cancers worldwide. Unfortunately, the prognosis of PDAC is rather poor and, for instance in the USA, over 47,000 people die because of pancreatic cancer annually. The present invention involves the discovery that high expression of acid sphingomyelinase (ASM) in PDAC strongly correlates with long-term survival of patients, as revealed by the analysis of two independent data sources. The positive effects of acid sphingomyelinase expression on long-term survival of PDAC patients was independent of patient demographics as well as tumor grade, lymph node involvement, perineural invasion, tumor stage, lymphovascular invasion and adjuvant therapy. In addition, the present invention has found that genetic deficiency or pharmacological inhibition of the acid sphingomyelinase promotes tumor growth in an orthotopic mouse model of PDAC. This is mirrored by a poorer pathologic response, as defined by the College of American Pathologists (CAP) score for pancreatic cancer, to neoadjuvant therapy of patients co-treated with functional inhibitors of the acid sphingomyelinase, in particular tricyclic antidepressants and selective serotonin reuptake inhibitors, in a retrospective analysis. The data disclosed herein indicate expression of the acid sphingomyelinase in PDAC as a prognostic marker for tumor progression. They further suggest that the use of functional inhibitors of the acid sphingomyelinase, at least of tricyclic antidepressants and selective serotonin reuptake inhibitors in patients with PDAC is contra-indicated. Finally, the data also support one embodiment of the present invention, a novel treatment of PDAC patients with recombinant acid sphingomyelinase.

In one embodiment, the present invention is a method for predicting the survival time of a patient suffering from pancreatic cancer. The method involves the steps of 1) measuring acid sphingomyelinase expression in a tumor sample obtained from the patient, 2) categorizing the level of acid sphingomyelinase expression as high or low, and 3) using the level of acid sphingomyelinase expression to predict survival time of the patient. In one embodiment, a high level of acid sphingomyelinase expression indicates an increased likelihood of long-term survival and an improved prognosis for the patient. In another embodiment, a low level of acid sphingomyelinase expression indicates a decreased likelihood of long-term survival and a poor prognosis for the patient.

As an example, the measurement of acid sphingomyelinase expression is conducted as follows:

IHC scoring a formalin fixed tissue. The slides were then scored on an ordinal scale based on the intensity of acid sphingomyelinase staining: 0=negative or no staining, 1+=weak staining, 2+=intermediate/moderate staining, and 3+=strong staining intensity. Patients were then categorized as ASM low expression (staining intensity of 0 or 1+) or ASM high expression (staining intensity of 2+ or 3+) for comparison. RNA sequencing score is determined by RNA expression levels and based optimal cut point expression levels.

The data presented herein indicate an important role of the acid sphingomyelinase for progression of PDAC and therapy of patients with PDAC. The data obtained from two independent tissue banks and patient groups indicate that a high expression of the acid sphingomyelinase in the tumor tissue strongly correlates with long-term survival of PDAC patients. These data are based, at least for the patient group from the University of Cincinnati, on tissue samples from untreated patients. These patients were on a surgery first approach and the malignant tumor tissue was removed prior to any chemotherapy, indicating that expression of the acid sphingomyelinase in the tumor tissue is a true prognostic marker for PDAC. These data show that the acid sphingomyelinase serves as a novel marker to predict tumor progression in patients with PDAC. The acid sphingomyelinase may also serve as marker to determine whether a patient requires more or less aggressive treatment.

The patient data were obtained from staining of tumor specimen and analysis of the acid sphingomyelinase in the malignant tumor cells. The other data sets were obtained from the Human Protein Atlas and was based on biopsies. It reflects mRNA expression of the acid sphingomyelinase in these samples without specification of any cell type. The data was obtained in acid sphingomyelinase-deficient mice that were injected with Pan02 pancreas carcinoma cells. It suggests that not only the expression of the acid sphingomyelinase in tumor cells is important for tumor progression, but also the expression of the acid sphingomyelinase in host cells. In our orthotopic tumor model in knock-out mice, the acid sphingomyelinase is deficient in host cells, but not in the malignant tumor cells. Thus, these data suggest that expression of the acid sphingomyelinase in the malignant and non-malignant tumor cells determine prognosis of the patients.

The data indicate that inhibition of the acid sphingomyelinase in PDAC patients may result in faster tumor growth and/or reduced response to chemotherapy. Tricyclic antidepressants and selective serotonin reuptake inhibitors (SSRI) inhibit the acid sphingomyelinase. These antidepressants induce the release of acid sphingomyelinase from lysosomal membranes, thereby triggering the degradation of the enzyme in the lysosomal lumen. In detail, the acid sphingomyelinase seems to predominantly associate with intralysosomal membranes and the interaction of the enzyme with these membranes is targeted by drugs such as antidepressants. Antidepressants inhibiting the acid sphingomyelinase are weak bases that are protonated in lysosomes and thereby trapped in lysosomes. The organic ring system of these compounds may bind to lipid membranes, whereas the protonated tertiary amine displaces acid sphingomyelinase from lysosomal membranes and thereby induces degradation of the acid sphingomyelinase. Thus, these weak bases do not directly inhibit acid sphingomyelinase activity but rather functionally inhibit the enzyme.

The data indicate that inhibition of the acid sphingomyelinase in patients treated with tricyclic antidepressants or SSRI results in increased tumor progression and a reduced response to neoadjuvant treatment. These data are clinically very important and suggest that patients with PDAC should not be treated with FIASMA-antidepressants or other compounds that inhibit the acid sphingomyelinasc.

The data presented herein show that expression of the acid sphingomyelinase correlates with long-term survival of pancreas cancer patients. The data suggest the acid sphingomyelinase as a novel marker for pancreas cancer prognosis. They also indicate that patients with pancreas cancer should not be treated with pharmacological inhibitors of the acid sphingomyelinase, such as many antidepressants. Finally, this data shows that treatment of patients with PDAC with recombinant acid sphingomyelinase to increase endogenous levels of acid sphingomyelinase expression may serve to develop novel treatments of PDAC.

### Treatment Method

In one embodiment, the present invention is a novel treatment for pancreatic cancer based on the role of acid sphingomyelinase (ASM) activity in tumorigenesis and immune regulation. This aspect of the present invention focuses on ASM activity and alterations in lysosomal sphingomyelin and their role of regulating lysosome metabolism via its ability to regulate arginine uptake coupling sphingolipids to mTORC1 signaling, arginase-1 (Arg-1) and arginine decarboxylase (ADC) expression and ultimately immune regulation in response to PDAC. One embodiment of the present invention is a novel PDAC treatment strategy targeting/reconstituting the lysosomal ASM-sphingomyelin-Arg-1/ADC axis by administering recombinant ASM.

In one embodiment, a therapeutic agent comprising recombinant ASM is administered to a patient for treatment of pancreatic cancer. The therapeutic agent may further comprise modified enzymes, fusion proteins, constitutively active mutants, etc. A therapeutic agent described herein that comprises recombinant ASM is administered to a subject at a therapeutically effective amount or dose. In some embodiments, the therapeutically effective concentration of the agent is a concentration that treats one or more symptoms of pancreatic adenocarcinoma in the individual. In some embodiments, the therapeutic agent comprising recombinant ASM is administered in conjunction with current cancer treatments such as chemotherapy, radiation therapy, immunotherapy, including adoptive immunotherapy therapy with TIL (Tumor Infiltration Lymphocytes), and bone marrow transplantation.

Illustrative dosages include a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In some embodiments, a patient initially is given a low dose, which is then increased to an efficacious dosage tolerable to the patient. Determination of an effective amount is well within the capability of those skilled in the art.

In various embodiments, a therapeutic agent described herein is administered parenterally. In some embodiments, the therapeutic agent is administered intravenously. Intravenous administration can be by infusion, e.g., over a period of from about 10 to about 30 minutes, or over a period of at least 1 hour, 2 hours, or 3 hours. In some embodiments, the therapeutic agent is administered as a continuous infusion. In some embodiments, the therapeutic agent is administered as an intravenous bolus. Combinations of infusion and bolus administration may also be used.

In some parenteral embodiments, a therapeutic agent is linked to an engineered polypeptide, peptide, or antibody. In some embodiments, the therapeutic agent is administered intraperitoneally, subcutaneously, intradermally, or intramuscularly. In some embodiments, the agent linked is administered intradermally or intramuscularly. In some embodiments, the agent is administered intrathecally, such as by epidural administration, or intracerebroventricularly.

In other embodiments, a therapeutic agent may be administered orally, by pulmonary administration, intranasal administration, intraocular administration, or by topical administration. Pulmonary administration can also be employed, e.g., by use of an inhaler or nebulizer, and formulation with an acrosolizing agent.

### EXAMPLES

An investigation was conducted in three independent patient cohorts and in pharmacological and genetic mouse models the role of the acid sphingomyelinase for the prognosis of pancreas cancer. The acid sphingomyelinase-ceramide system has been implied in the pathophysiology of malignant tumors, but its role in pancreas cancer is unknown. To analyze whether expression of the acid sphingomyelinase plays a role in human pancreatic ductal adenocarcinoma (PDAC), the expression of the acid sphingomyelinase in tissue samples were correlated from patients that were diagnosed with resectable PDAC, but not yet treated with any chemotherapy or surgical intervention, with the long-term survival of these patients. The patients underwent a surgery first approach, i.e. surgery was performed prior to any other treatment, and therefore we were able to obtain tumor specimen for histology analysis prior to any chemotherapy. Clinical data including patient demographics, clinical, operative and pathologic characteristics, as well as survival was determined (Tables 2 and 3).

### Example 1

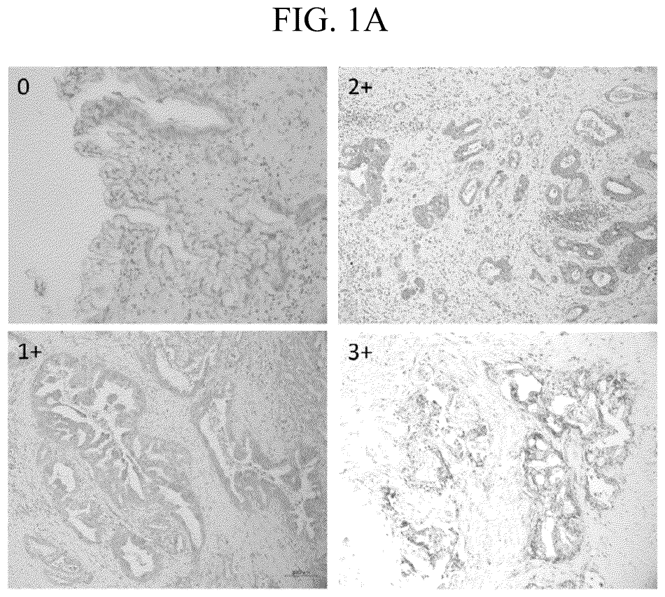

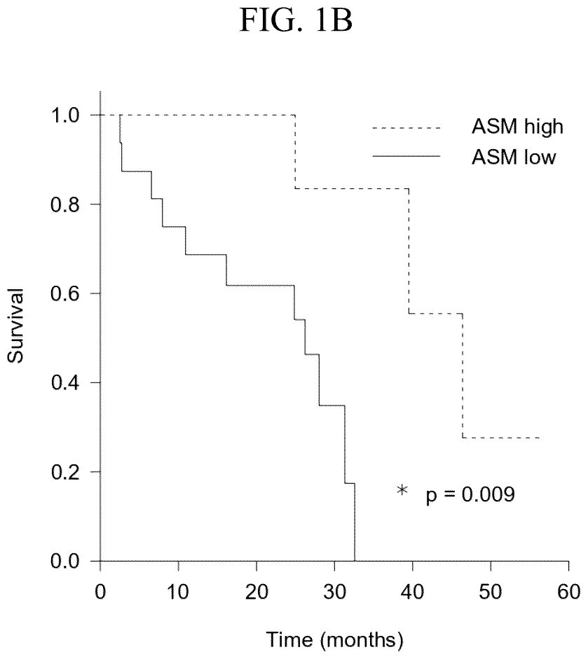

The immunohistochemistry analysis of acid sphingomyelinase expression was graded from 0 (no detectable acid sphingomyelinase expression) to 3+ (strong expression of the acid sphingomyelinase) as shown in FIG. 1A. Correlation of the acid sphingomyelinase expression with patient survival data indicates that high expression of acid sphingomyelinase in the tumor tissue strongly correlated with improved prognosis of the patients with overall survival (FIG. 1B). Tumors with 2+ or 3+ staining for SMPDI were categorized as SMPDI high expression while those with 0 or 1+ staining were categorized at SMPDI low expression.

Referring to FIG. 1A, IHC staining for acid sphingomyelinase was performed on human PDAC resection specimens (n=23). All patients underwent a surgery-first approach with no preoperative therapy. Shown are representative examples from the 23 specimens. Referring to FIG. 1B, overall patient survival by ASM expression. Median overall survival was 26.3 months in the acid sphingomyelinase low cohort and 46.4 months in the acid sphingomyelinase high cohort. Low expression was defined as 0 or 1+ tumor staining and high expression as 2+ or 3+ staining. Overall survival was evaluated using the Kaplan-Meier estimator, for statistical analysis the log-rank test was used.

Age, sex and race, the type of resection, tumor grade, lymph node involvement, perineural invasion, tumor stage, lymphovascular invasion and adjuvant therapy did not differ between the groups with high and low expression of the acid sphingomyelinase (Tables 2 and 3).

The analysis revealed that a high expression of the acid sphingomyelinase strongly correlated with the prognosis and overall survival of the patients.

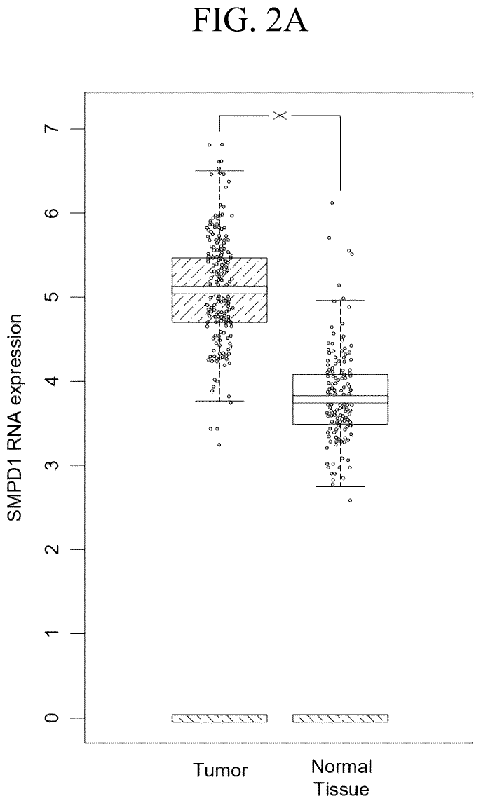

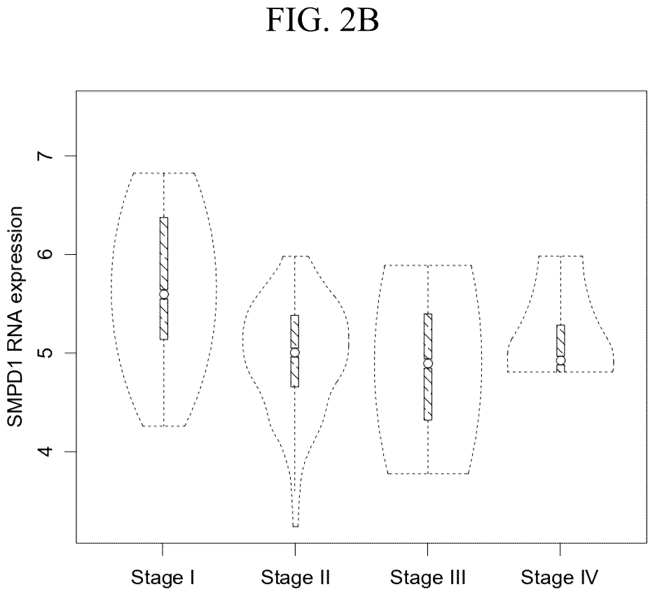

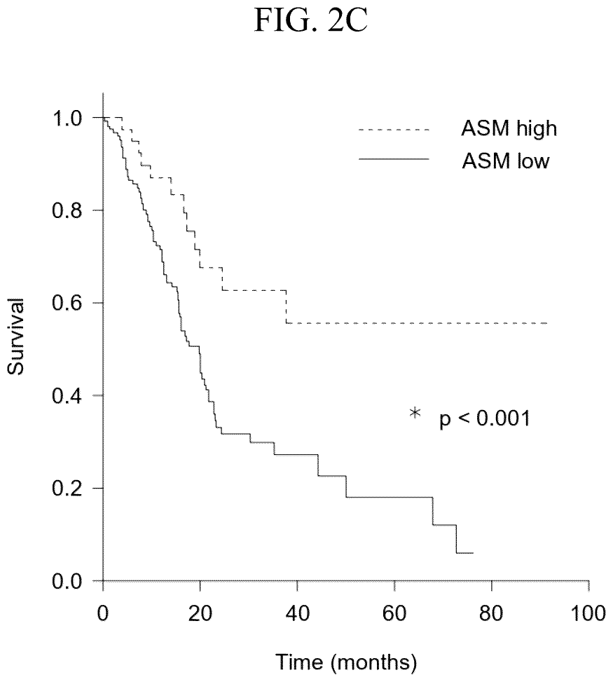

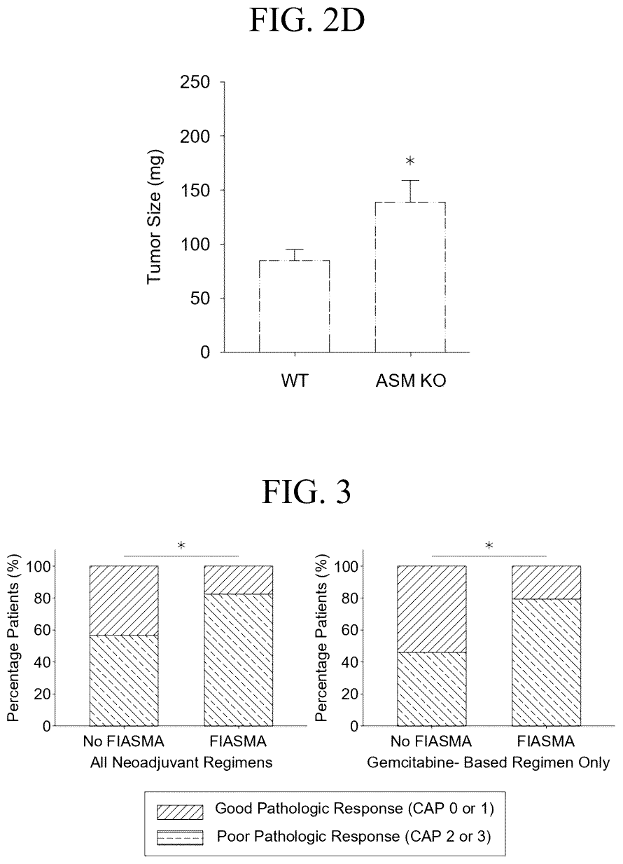

To confirm these data, we analyzed data from the Human Protein Atlas (proteinatlas.org) and the publicly available GEPIA database (Gene Expression Profiling Interactive Analysis). We compared mRNA expression levels of SMPDI in both tumor (TCGA) and normal tissue samples (from TCGA+Gtex). The studies revealed a higher overall expression of the enzyme in tumor samples with respect to normal tissues (FIG. 2A). This observation was confirmed also in a separate independent dataset, CPTAC, that contains pancreatic ductal adenocarcinoma tissue from 137 patients and 74 normal adjacent tissues. We also evaluated the expression levels of SPMDI in pancreatic adenocarcinoma stratified by stage on the basis of the TCGA dataset, in order to take into consideration cancer aggressiveness. Interestingly, SPMDI expression level found to be significantly higher in stage 1 but similarly elevated in more advanced stages of pancreatic ductal adenocarcinoma tumor stage (F-value=7.9; Pr(>F)=5,76e-05) (FIG. 2B).

FIG. 2A is a graph showing the mRNA expression of SMPDI, which was assessed comparing tumor (red) and normal tissue (grey) from TCGA and GTEx datasets on the GEPIA database. Data were normalized as transcripts per kilobase million (TPM) values. TPM values were converted to log 2-normalized transcripts per million [log 2(TPM+1)]. Data were shown as the mean±standard deviation. Statistical analyses were performed using t-test. Error bars represented SD. *: p-value<0.05. Similar findings were found in the proteogenomic characterization of SMPD1 in pancreatic adenocarcinoma and adjacent normal pancreas tissue from the CPTAC samples (data not shown). FIG. 2B is a violin plot of acid spingomyelinase (SMPD1) expression stratified by pancreatic cancer stage from the TCGA dataset. Values were normalized as transcripts per kilobase million (TPM) values. TPM values were converted to log 2-normalized transcripts per million [log 2(TPM+1)]. Statistical analyses were performed using Fisher's exact test. F value=7.9, Pr(>F)=5.76e-05.

These results justify further analysis of the acid sphingomyelinase expression in tumor specimen and, thus, we correlated acid sphingomyelinase expression, based on RNA sequence data (Human Protein Atlas dataset), from 172 patients with PDAC with the long-term survival of the patients. Overall median expression of SMPDI was 17.62 FPKM. Optimal expression cutoff value based on survival analysis was identified as 22.99. Patient details including age and stage are listed in Table 4.

The results confirm the data obtained with our cohort and show a strong correlation between acid sphingomyelinase expression in the tumor and long-term survival of the patients (FIG. 2C) Similar survival results were seen even when all stage 1 patients were excluded from the survival analysis (not shown). FIG. 2C shows a survival analysis by acid sphingomyelinase expression in the human protein atlas database. RNA sequence data were from the open source Human Protein Atlas (proteinatlas.org); n=172 patients. All stage 4 patients were eliminated from the cohort. Median overall survival was not reached in the high expression cohort compared to 19.7 months in the SMPDI low expression group (p<0.001). Overall survival was evaluated with Kaplan-Meier survival curves with comparison made between the two groups by log-rank test.

Collectively, the data presented herein indicate that expression of the acid sphingomyelinase in PDAC strongly correlates with the prognosis and long-term survival of the patients. Low expression of the acid sphingomyelinase correlates with a poor prognosis of PDAC patients.

### Example 2

It has been previously shown that antidepressants and other medications inhibit the acid sphingomyelinase by displacing the enzyme from lysosomal membranes resulting in the degradation of the acid sphingomyelinase within lysosomes. This raises the question whether the application of antidepressants or other functional inhibitors of acid sphingomyelinase (FIASMA) impacts the prognosis of patients with PDAC. We analyzed a consecutive patient cohort with PDAC treated with neoadjuvant therapy followed by surgery at the University of Cincinnati. The patients were categorized by the use of functional inhibitors of ASM during the neoadjuvant treatment period. We then assessed the response to treatment by analyzing resection specimens. A good pathologic response was defined by a CAP score of 0 or 1 and was seen in 43.5% of patients not co-medication with a functional inhibitor of the acid sphingomyelinase compared to 17.9% of patients on a FIASMA co-medication during ncoadjuvant therapy (FIG. 3), suggesting that inhibition of the acid sphingomyelinase inhibits the tumor response to neoadjuvant treatment. FIG. 3 is a pair of graphs showing that functional inhibitors of acid sphingomyelinase during neoadjuvant treatment of PDAC impact prognosis of patients. 94 consecutive patients treated with neoadjuvant therapy followed by surgery at the University of Cincinnati were examined. Patients were categorized by the use of functional inhibitors of acid sphingomyelinase (FIASMA) during the neoadjuvant treatment period and were assessed for pathologic response to treatment on the resection specimens. Pathologic response in surgical specimens was graded based on the proportion of viable tumor according to College of American Pathologist (CAP) grading system. Grade 0 (complete histologic response, no viable cancer cells) and 1 (near complete response, single cells or rare small groups of cancer cells) specimens were categorized as a good pathologic response, while grades of 2 (partial response with residual cancer) and 3 (poor or no response with extensive residual cancer) were categorized as a poor pathologic response. A good pathologic response (CAP score of 0 or 1) was seen in 43.5% of patients not on a functional inhibitor of the acid sphingomyelinase compared to 17.9% of patients on a functional inhibitor of the acid sphingomyelinase during neoadjuvant therapy. Similar findings were seen in the subset of patients that were treated with gemcitabine-based chemotherapy (good pathologic response scen in 54% of patients not on a functional inhibitor of acid sphingomyelinase compared to 20% of patients on a functional inhibitor of acid sphingomyelinase). *p<0.05. Pathologic responses were compared between cohorts with the Fisher Exact test.

### Example 3

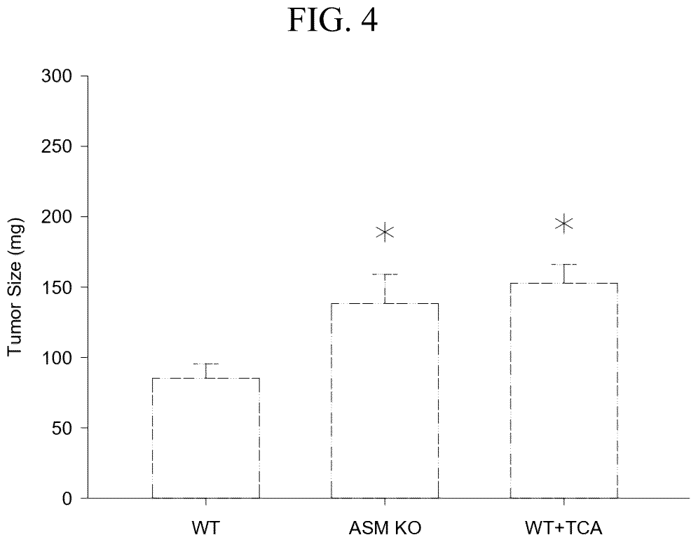

To further prove the notion that a down-regulation of the acid sphingomyelinase in the tumor tissue regulates tumor progression, we established orthotopic pancreas cancers in wildtype mice and in acid sphingomyelinase-deficient mice. The wildtype mice were randomly divided into 2 groups treated with antidepressant (amitriptyline 10 mg/kg, i.p. injected every 2nd day) or vehicle, i.e. 0.9% NaCl. The size of the pancreas cancer was determined 15 days after tumor initiation. The results show that PDAC grows much faster in mice lacking the acid sphingomyelinase compared to wildtype mice (FIG. 4). Even more importantly, the treatment of wildtype mice with antidepressants at doses that result in therapeutic blood levels also resulted in increased tumor growth (FIG. 4).

FIG. 4 is a graph showing a growth of PDAC in murine models of orthotopic pancreatic ductal adenocarcinoma. Wildtype mice were injected orthotopically into the pancreas with 106 Pan02 pancreas cancer cells and either left untreated or treated with amitriptyline at 10 mg/kg every other day via IP injection starting at day 4 after tumor injection. In addition, we injected acid sphingomyelinase-deficient littermates with Pan02 pancreas cancer cells orthotopically into the pancreas. Tumor size was determined 15 days after injection. Shown are the mean±SD of the tumor size from each n=4-5 mice per group; *p<0.05, ANOVA with Tukey post hoc test.

### Example 4

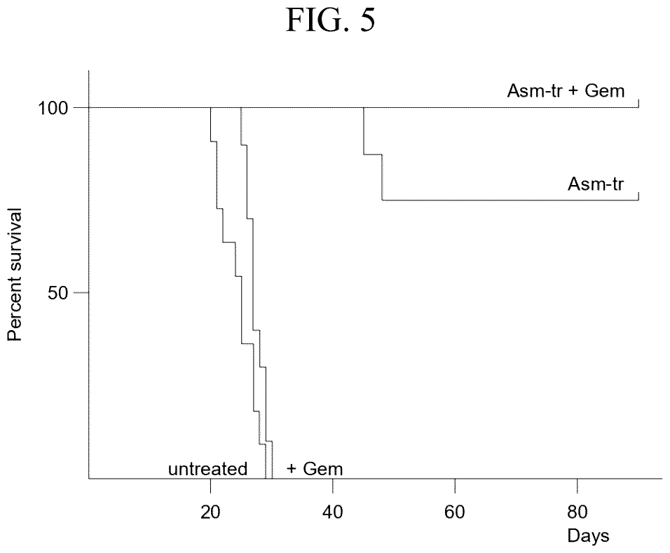

An experiment was conducted to determine if acid sphingomyelinase overexpression prevents tumor growth. Based on the clinical data, the effects of ASM overexpression and tumor growth were evaluated. Intraperitoneal injection of KPC-PDAC cells resulted in multiple tumors in wt mice within 3 to 4 weeks; these tumors were resistant to treatment with gemcitabine (FIG. 5). In marked contrast, only 20% of ASM-transgenic (ASM-tr) overexpressing mice developed a tumor, and treatment of ASM-tr mice with a low dose of gemcitabine was sufficient to completely eradicate the tumors. Additional control experiments demonstrated that the early establishment and peritoneal growth of KPC cells was similar in wt and ASM-tr mice up to 5 days after injection. This finding suggests that the ASM in host cells plays a central role in the control of pancreatic cancer.

### Example 5

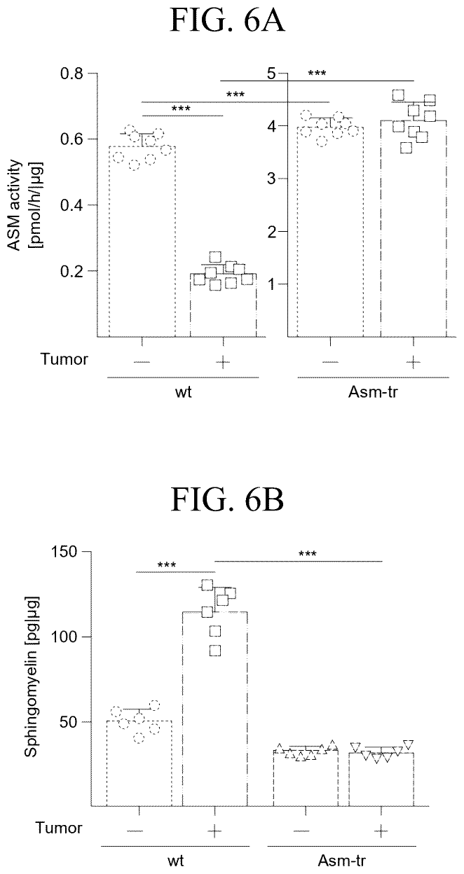

It was next tested whether PDAC alters the ASM/sphingomyelin system in immune cells of the spleen and bone marrow. Because tumors did not develop in most ASM-tr mice, we did not investigate the local tumor immune cells. Mice were given i.p. injections of KPC cells. After 2 weeks, the spleen was removed, and ASM activity and sphingomyelin concentrations were determined. The results demonstrate an inhibition of ASM activity and an accumulation of sphingomyelin in the spleen and the bone marrow of wt mice as early as 7 or 14 days after i.p. injection of tumor cells (FIGS. 6A and 6B). Splenocytes or bone marrow cells from ASM-tr mice displayed approximately 10-fold higher ASM activity, and no accumulation of sphingomyelin was detected in these cells 7, 14, or 28 days after i.p. tumor injection (FIGS. 6A and 6B). Measurements of ASM activity in macrophages, polymorphonuclear neutrophils (PMNs), T-cells, and B-lymphocytes sorted from the spleen confirmed the inhibition of ASM and the accumulation of sphingomyelin in wt cells as early as 7 or 14 days after tumor injection. These results indicate that PDAC induces inhibition of acid sphingomyelinase in immune cells in vivo.

FIG. 6A is a graph showing ASM activity and sphingomyelin levels in spleens of wildtype (wt) or ASM-tr mice 14 days after i.p. injection of KPC cells or of vehicle control. ASM activity in cell homogenates was measured by consumption of [¹⁴C]sphingomyelin to ceramide and [¹⁴C]phosphorylcholine, which was scintillation counted after phase separation. Sphingomyelin was quantified by a commercial ELISA. Mean±SD; n=6-8; ***p<0.001 ANOVA

### Example 6

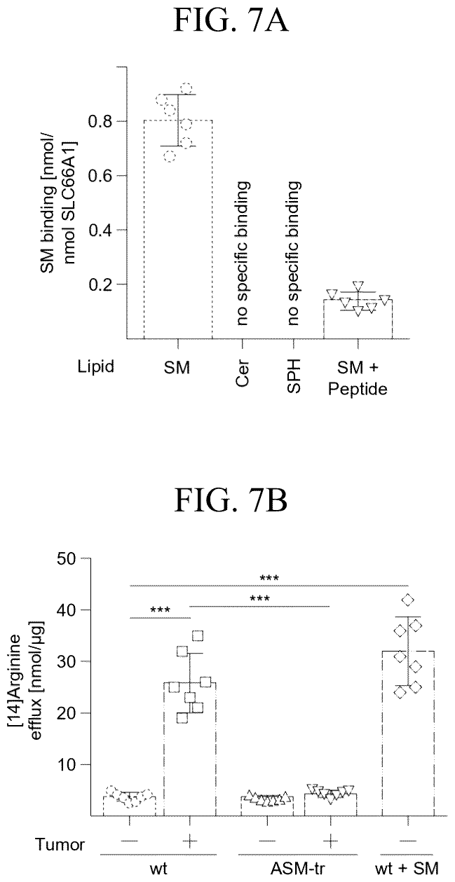

Sphingomyelin has been shown to bind to a motif consisting of VXXTLXXIY. The lysosomal arginine transporter SLC66A1 contains a VMLTLYFYY motif. A study was conducted to investigate whether SLC66A binds to sphingomyelin. Our immunoprecipitation experiments showed direct and specific binding of sphingomyelin to SLC66A1, which is almost abrogated in the presence of excess synthetic VMLTLYFYY peptide (FIG. 7A). Additionally, efflux of arginine was markedly higher in lysosomes of splenic myeloid cells isolated from wt tumors than in cells from control mice and absent in ASM-tr cells (FIG. 7B). Furthermore, sphingomyelin strongly activated the efflux of arginine from lysosomes of wt cells (FIG. 7B). These results indicate that lysosomal sphingomyelin controls lysosomal arginine transport.

FIG. 7A is a graph showing SLC66A1 was immunoprecipitated from 40×10⁶ splenocytes, after lysis in RIPA-buffer+aprotinin/leupeptin. Immunocomplexes were immobilized on protein A/G agarose, washed 3-times in RIPA buffer and twice in PBS+0.1% Triton X100, resuspended in the same buffer and binding of [¹⁴C]sphingomyelin (SM), [¹⁴C]ceramide (Cer), or [¹⁴C]sphingosine (SPH) was measured in the absence or presence of the VMLTLYFYY peptide.

FIG. 7B is a graph showing isolated lysosomes from Ly6G-sorted splenocytes obtained from mice with or without PDAC were loaded with [¹⁴C]arginine and the efflux of [3H]arginine was determined. Aliquots of the lysosomes were treated with sphingomyelin. Mean±SD; n=6-8; ***p<0.001 ANOVA.

### Example 7

It has been shown that lysosomal arginine concentrations control the association of mTORC1 with lysosomal membranes and, via mTORC1, the activity of TFEB, a transcription factor that controls expression of Arg-1. Arg-1 converts arginine in the cytoplasm and thereby controls lymphocyte activity, because these cells require sufficient arginine levels for activation and survival. ADC also controls arginine levels, but it is unknown whether this enzyme is also involved in immune regulation.

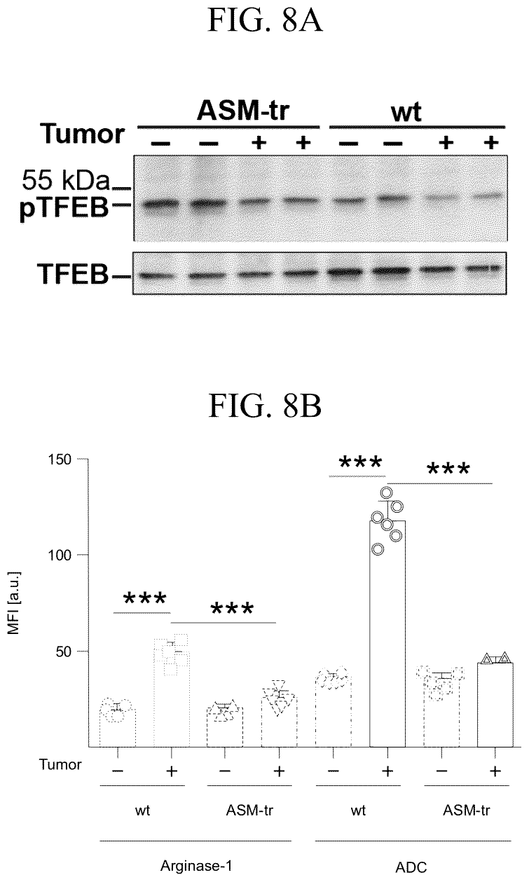

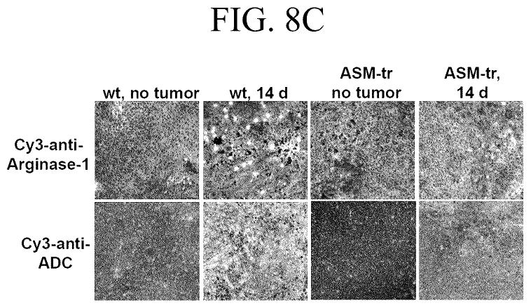

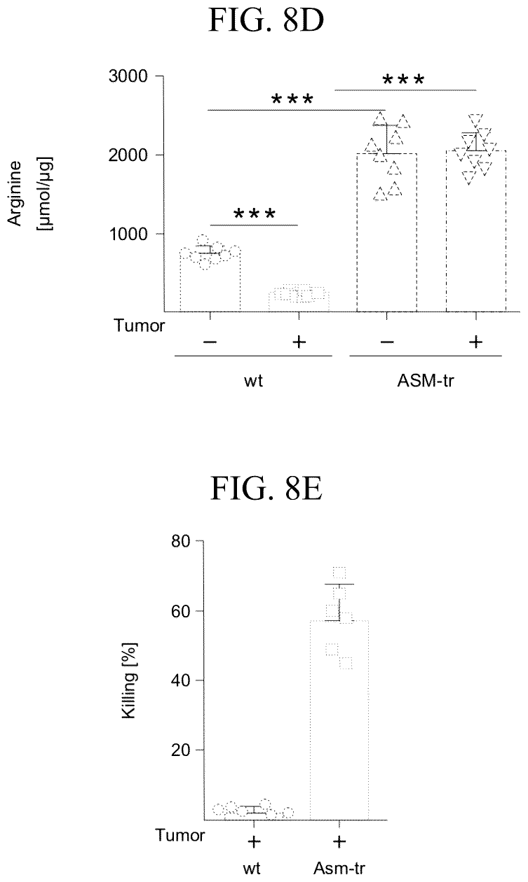

Our findings show that peritoneal PDAC induces the activation of TFEB (measured by its de-phosphorylation) (FIG. 8A), the translocation of TFEB into the nucleus, an increase in the expression of Arg-1 and ADC, and a concomitant decrease in arginine concentrations in the spleen of wt mice (FIGS. 8B and 8D). Overexpression of ASM prevented these events (FIGS. 8A-8D). Similar results were found in isolated splenic Ly6G- or F4/80-positive myeloid cells and in bone marrow cells. It has been shown that Arg-1 suppresses the immune system, because arginine is required for T-lymphocyte activation.

Further, it was confirmed that sphingomyelin caused upregulation of Arg-1 and ADC and the concomitant reduction of arginine concentrations results in immune inhibition. Confirmation was accomplished by the finding that anti-CD3-mediated IL-2 and INFγ-formation, as well as cytotoxicity toward KPC cells, is lower in splenocytes isolated from wt mice injected with PDAC than in splenocytes from ASM-tr mice (FIG. 8E). Adding arginine to isolated immune cells from tumors in wt mice restored CD3-mediated formation of IL-2 and INFγ, a finding suggesting that the immune defect in tumor wt mice is mediated by an increase in Arg-1 and ADC expression. These results indicate that lysosomal sphingomyelin controls mTORC1 and TFEB concentrations, Arg-1/ADC expression, and arginine levels in immune cells.

### Example 8

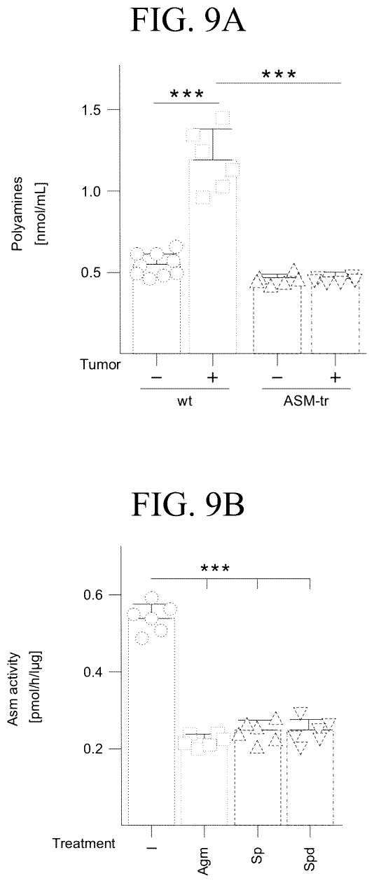

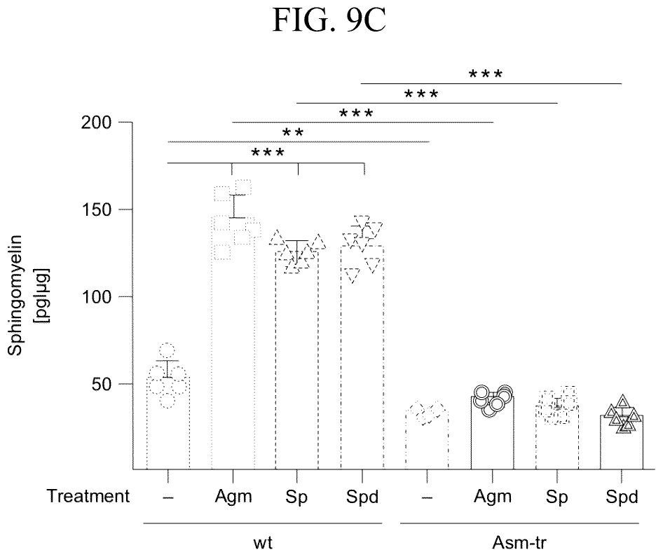

An increase of Arg-1 and ADC expression may also increase polyamine concentrations. Measurements of polyamine concentrations in the blood plasma of wt mice indicated an increase in polyamine concentrations in the spleen after tumor injection; this increase was absent from ASM-tr mice (FIG. 9A). Polyamine concentrations were also increased in the blood plasma of patients with pancreatic cancer, consistent with previous publications. Thus, we tested whether polyamine concentrations alter ASM activity. Incubation of ex vivo splenocytes, Ly6G-sorted myeloid cells, or cultured J774 macrophages with the polyamines agmatine (produced by both enzymes) inhibited ASM activity and increased cellular sphingomyelin concentrations, an increase that was absent from cells overexpressing ASM (FIGS. 9B and 9C). Since ASM-tr mice usually do not form tumors, we co-cultured KPC-pancreas cancer cells with splenocytes and immune cells isolated from peritoneal lymph nodes which confirmed inhibition of the acid sphingomyelinase and accumulation of sphingomyelin in immune cells in the local environment with PDAC as well. These results indicate that polyamines inhibit acid sphingomyelinase activity.

### Example 9

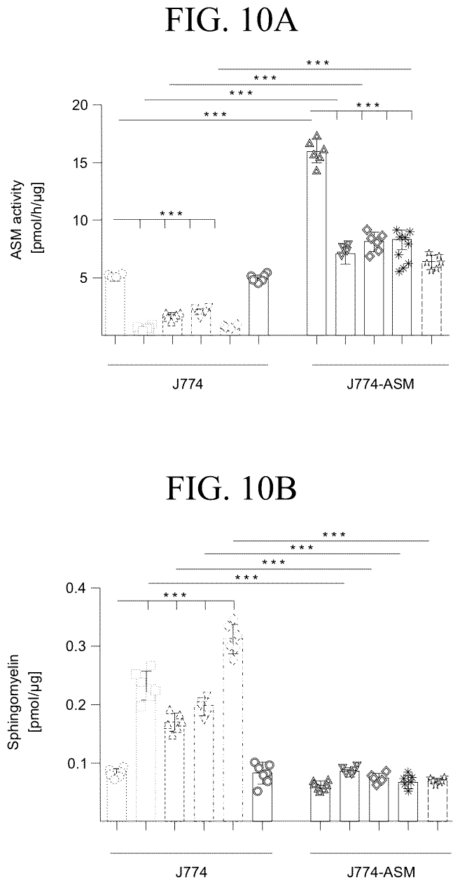

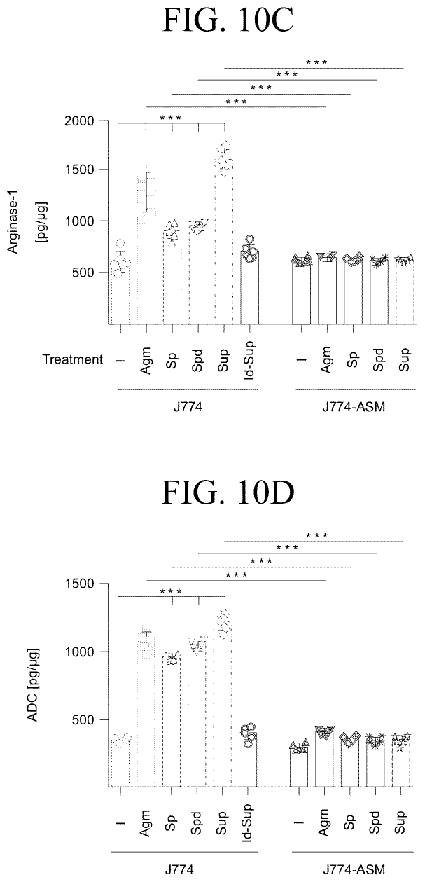

These findings suggest a vicious cycle of Arg-1/ADC expression, arginine consumption, and polyamine synthesis-further driving the inhibition of ASM-resulting in an immunosuppressive tumor environment. However, polyamines are not only produced by immune cells in wt mice challenged with PDAC; they are also produced by malignant cells. Our results confirm that KPC cells release polyamines, but this release was reduced by transfection of KPC cells with ASM. Incubation of J774 macrophages with culture supernatant from KPC cells inhibited ASM activity and sphingomyelin accumulation and also increased Arg-1 and ADC expression in J774 cells (FIGS. 10A-10D). Immunodepletion of agmatine, spermine, and spermidine from KPC-derived supernatants or overexpression of ASM in J774 cells prevented these events (FIG. 10), a finding suggesting that polyamines mediate these events via ASM. These results indicate that polyamines produced by malignant tumor cells initiate immune inhibition in pancreas cancer.

FIG. 10 is a graph showing ASM activity (A) and levels of sphingomyelin (B), Arg-1 (C), and ADC (D) were determined in macrophages (J774 cell line) or in stable ASM-transfected J774 (with an approximately 3-fold overexpression of ASM) at baseline, after treatment with agmatine (Agm, 5 μM), spermine (Sp, 100 μM), or spermidine (Spd, 100 μM), supernatant (Sup) from KPC cells, and supernatant immunodepleted (id) of polyamines. ASM activity was measured by determining the consumption of [¹⁴C]sphingomyelin, Sphingomyelin-, Arginase-1- and ADC-levels were quantified by ELISA. Mean±SD; n=6-8; ***p<0.001, ANOVA

### Example 10

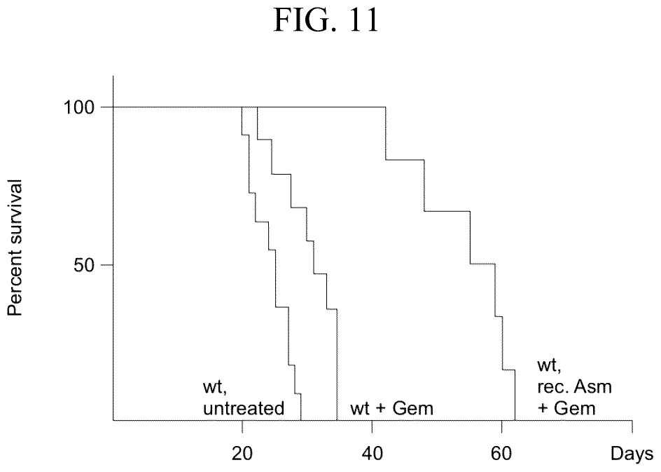

Since PDAC growth in ASM-tr mice. we determined the therapeutic potential of exogenous administration of recombinant ASM. We administered 0.75 mg/kg recombinant ASM i.v. twice daily to tumor-injected mice for 2 treatment cycles of 4 days each in combination with the standard treatment, i.e. gemcitabine. This administration delayed tumor growth (FIG. 11), whereas control mice exhibited massive tumors as early as 25 days after tumor injection. However, because the half-life of recombinant ASM is only 2 h, these studies must be repeated with continuous infusion of ASM. These results indicate that exogenous ASM can be used as a potential treatment.

Wt mice were treated with gemcitabine alone (“Group 1”), gemcitabine plus recombinant ASM (“Group 2”) or vehicle control after peritoneal injection of KPC cells (“Group 3”). Recombinant ASM was administered twice daily 0.75 mg/kg i.v. for two treatment cycles of 4 days each. The results are shown in FIG. 11.

### Example 11

Survival data was collected for three test groups (shown in Table 1).

Group 1: 50 000 KPC tumor cells were i.p. injected and tumor development was determined. For this group, the subjects survived, respectively, for 27, 28, 28, 29, 30, and 31 days.

Group 2: Survival time was collected for subjects upon application of recombinant acid sphingomyelinase (i.p., twice daily, for 2 weeks, starting 2 days after tumor injection). For this group, the subjects survived, respectively, for 48, 55, 57, 60, 62, 65 and 70 days. The acid sphingomyelinase was injected for a longer time and i.p. in these experiments than in FIG. 11.

Group 3: Survival time was collected for subjects upon application of recombinant acid sphingomyelinase (i.p., twice daily, for 2 weeks, starting 2 days after tumor injection)+application of anti-PD1 antibodies (every 2nd day i.p., 250 μg/25 g). For this group, the subjects survived, respectively, for 75, 80, 85, 87, 90, 93 and 96 days. The half-life time of acid sphingomyelinase is rather short in mice (2 hrs, but much longer in humans with 2 weeks). Thus, the i.p. injection are not very sufficient and must be replaced by continuous infusion, which may allow complete elimination of the tumors.

Survival data was collected for three test groups (shown in Table 1).

TABLE 1

| | Subject | Subject | Subject | Subject | Subject | Subject | Subject | Avg. |
|---|---|---|---|---|---|---|---|---|
| Group | 1 | 2 | 3 | 4 | 5 | 6 | 7 | Survival |
| 1 untreated | 27 | 28 | 28 | 29 | 30 | 31 | | 28.8 |
| 2 Rec. | 48 | 55 | 57 | 60 | 62 | 65 | 70 | 59.6 |
| ASM alone | | | | | | | | |
| 3 Rec. | 75 | 80 | 85 | 87 | 90 | 93 | 96 | 86.6 |
| ASM anti- | | | | | | | | |
| PD1 | | | | | | | | |

### Example 12

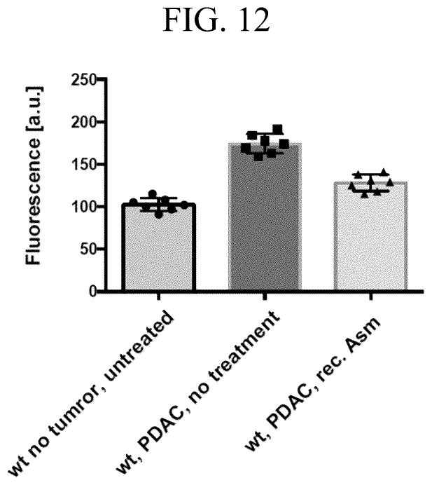

The PD1 expression on lymphocytes with/without treatment with recombinant acid sphingomyelinase was determined. The surface expression of PD1 on CD3 lymphocytes was determined by flow cytometry upon staining with FITC-anti-PD1 and APC-anti-CD3 antibodies, expressed as mean fluorescence in a.u. of PD1 in the CD3-positive cell population. Mice were i.p. injected with 50 000 KPC tumor cells, left for 21 days untreated or treated with recombinant acid sphingomyelinase for 2 days with 4 i.p. injections/day. The results are shown in FIG. 12.

Wildtype mice, no tumor, no treatment: 105/100/115/97/102/108/91 a.u.

Wildtype mice, KPC-PDAC, no treatment: 163/184/159/174/178/169/192 a.u.

Wildtype mice, KPC-PDAC, injection of rec. Asm: 138/129/125/118/131/115/141

### Methods

### Patient Cohorts

University of Cincinnati Medical Center Cohort Surgery-First Cohort: Consecutive patients (n=23) with resectable pancreatic ductal adenocarcinoma (PDAC) undergoing a surgery-first approach from 2014-2019 with available paraffin-embedded tissue blocks were included for analysis. Only patients undergoing a surgery first approach were included for analysis in order to evaluate inherent baseline expression of acid sphingomyelinase within the tumor. Patient charts were reviewed and patient demographic, clinical, operative and pathologic data were collected.

### Human Protein Atlas Cohort

The human protein atlas (proteinatlas.org) is a publicly available data source licensed under the Creative Commons Attribution-ShareAlike 3.0 International License that includes a pathologic atlas of the human cancer transcriptome. The database was queried for mRNA expression of the sphingomyelin phosphodiesterase 1 (SMPDI) gene in patients with pancreatic ductal adenocarcinoma. SMPDI expression levels and clinical characteristics of 172 patients were collected which included survival and cancer stage. Stage 4 patients were excluded from the cohort for analysis.

### The Cancer Genome Atlas and Clinical Proteomic Tumor Analysis Consortium Cohort

The cancer genome atlas (TCGA) and Genotype-tissue expression (GTEx) TCGA is a freely web-based accessible database, which collects NGS data from more than 10,000 tumors across 33 cancer types until 2018. Gene expression and clinical data were taken into consideration in the present study. Genotype-tissue expression (GTEx) GTEx provides publicly available gene expression data from 53 normal tissue sites across nearly 1000 people by RNA sequencing. The Clinical Proteomic Tumor Analysis Consortium (CPTAC) dataset, publicly available since 2021, is based on whole-genome sequencing (WGS) and whole-exome sequencing (WES) of 140 pancreatic cancers with 67 normal adjacent tissues giving rise to proteogenomic characterization of pancreatic ductal adenocarcinoma. The cBioPortal for Cancer Genomics (http://www.cbioportal.org), GEPIA (gene expression profiling interactive analysis) (http://gepia.cancerpku.cn/) and the UALCAN database (http://ualcan.path.uab.cdu) analysis tools were employed.

### University of Cincinnati Neoadjuvant Cohort

Consecutive patients (n=94) with biopsy-proven pancreatic ductal adenocarcinoma that underwent neoadjuvant chemotherapy followed by surgical resection from 2012-2022 were included for analysis. Clinicopathologic data were collected from our institutional database. Medical reconciliation records were reviewed prior to, during and at the completion of neoadjuvant chemotherapy for all medications. Patients were then categorized based on the use of functional inhibitors of acid sphingomyelinase (FIASMA) during treatment. Pathologic response in surgical specimens was graded based on the proportion of viable tumor according to College of American Pathologist grading system. Grade 0 (complete histologic response, no viable cancer cells) and 1 (near complete response, single cells or rare small groups of cancer cells) specimens were categorized as a good pathologic response while grades of 2 (partial response with residual cancer) and 3 (poor or no response with extensive residual cancer) were categorized as a poor pathologic response.

The use and collection of all clinical and pathologic data and specimens was performed with approval and in accordance with the guidelines established by the University of Cincinnati Institutional Review Board (IRB 2019-0324).

### Immunohistochemical Staining

Acid sphingomyelinase staining protocol: Tissue slides were deparaffinized and rehydrated in xylene substitute and gradients of ethanol after heating for 1 hour at 65° C. Permeabilization was done with 1% Triton X-100 for 15 minutes on a shaker plate and then washed with PBS-Tween buffer. Antigen retrieval was done with Tris-EDTA, pH 9.0 buffer by using Thermo-Electron Corporation Shandon Tissue Wave 2, HIER 1 program. Slides were cooled to room temperature, washed with PBS-Tween buffer and then treated with 3% H2O2 for 1 h at 37° C. to block endogenous peroxidase. Specimens were then incubated with rabbit primary antibody, anti-Smpd1 (Proteintech, Cat #14609-1-AP) at 1:200 dilution at 4° C. overnight. The sections were extensively washed with PBS-Tween buffer and then incubated with biotinylated goat anti-rabbit secondary antibody at room temperature for 30 minutes. The specimens were washed with PBS-Tween buffer and then incubated with Streptavidin-HRP pre-diluted (SAV-HRP, #BD550946, ready to use) for 30 min at room temperature. The specimens were then treated with diaminobenzidine DAB (HRP substrate, ThermoScientific, TA-125-QHDX) about 1 min and the reaction stopped by transfer to distilled water. The specimens were then counterstained with hematoxylin. Sections were mounted onto slides with Permount (SP15-500, Thermo Fisher Scientific, USA) for review.

### Evaluation of Immunohistochemical Staining in Pathologic Specimens

All slides were reviewed and scored by a pathologist (JW) blinded to the identity of the samples. The pathologist is an expert in pancreatic cancer. Only staining within the invasive tumor component was considered when grading the staining. The slides were then scored on an ordinal scale based on the intensity of acid sphingomyelinase staining: 0=negative or no staining, 1+=weak staining, 2+=intermediate/moderate staining, and 3+=strong staining intensity. Patients were then categorized as ASM low expression (staining intensity of 0 or 1+) or ASM high expression (staining intensity of 2+ or 3+) for comparison.

### Orthotopic Pancreatic Cancer Murine Model

All animal experiments were approved by the University of Cincinnati Ethic Committee and the Institutional Animal Care and Use Committee. Eight-week-old, wild type male, C57BL/6J mice were purchased from Jackson Labs (000664, Jackson Labs, USA). Acid sphingomyelinase-deficient mice (Smpd1−/−) were bred and genotyped at the animal facility of the University of Cincinnati. We used young acid sphingomyelinase-deficient mice (maximum age 10 weeks) to avoid sphingomyelin accumulation. Mice were anesthetized using 120 mg/kg ketamine plus 20 mg/kg xylazine. Orthotopic injection was performed as described by Tepal et al. In detail, a left subcostal incision was made just below rib cage and the pancreas was identified. The tumor cell suspension was created by mixing 25 μL of Matrigel with 25 μL of Pan02 cells (National Cancer Institute-Frederick Cancer Research and Development Center, Frederick, MD, USA) containing 1×106 cells. Pan02 cells were cultured in DMEM+10% FBS medium, under 37° C. and 5% CO2, no antibiotic was added. Cells were washed, trypsinized and washed in PBS at least 3-times prior to injection. The tumor suspension was slowly injected into the pancreas and the needle left in place for 60 seconds to allow the Matrigel to set. After ensuring hemostasis, the abdomen was closed in 2 layers using 3-0 silk suture. Wildtype mice were then randomly divided into 2 groups. One group was treated with 10 mg/kg of the antidepressant and functional inhibitor of the acid sphingomyelinase amitriptyline or vehicle, i.e. 0.9% NaCl. Amitriptyline or 0.9% NaCl were injected into the peritoneal cavity every other day. The size of the pancreas cancer was determined in all groups 15 days after tumor initiation.

### Statistics

Clinicopathologic data were obtained from the electronic medical records and our institutional prospectively maintained pancreatic cancer database. Variables include patient age, gender, histologic grade, lymphovascular invasion, perineural invasion, stage, lymph node status, and oncologic status. Statistical analyses were performed using JMP Pro v15 (SAS, Cary, NC) and SigmaPlot 14.5 (Systat Software, Inc., Palo Alto, CA). Data were classified as categorical and continuous variables. Categorical variables were described with counts and percent with comparison made with Pearson chi-squared and Fischer exact test, as appropriate. Continuous variables were examined using Student's t-test. Survival analyses were performed using Kaplan-Meier methodology with log-rank test for group comparison. P-values <0.05 were considered statistically significant.

### Tables

The table indicates the patients' demographics of all patients included in the cohort analyzed at the University of Cincinnati. Continuous variables compared with student t-test. Categorical variables were compared with Fischer exact and Pearson chi-squared tests.

TABLE 2
Patient Demographics - Resectable Pancreatic Ductal Adenocarcinoma
undergoing surgery first approach treatment

| | All Patients | | SMPD1 Low | | SMPD1 High | | p-value |
|---|---|---|---|---|---|---|---|
| | 23 | | 69.6% | (n = 16) | 30.4% | (n = 7) | |
| Age, yrs | 69.3 +/− 1.5 | | 69.4 +/− 1.8 | | 69.1 +/− 3.2 | | 0.92 (student |
| (mean, SEM) | | | | | | | t-test) |
| Sex | | | | | | | 1.0 (Fischer |
| | | | | | | | Exact) |
| male | 60.9% | (n = 14) | 62.5% | (n = 10) | 57.1% | (n = 4) | |
| female | 39.1% | (n = 9) | 37.5% | (n = 6) | 42.9% | (n = 3) | |
| Race | | | | | | | 0.22 (pearson |
| | | | | | | | chi sq) |
| white | 87.0% | (n = 20) | 93.8% | (n = 15) | 71.4% | (n = 5) | |
| black | 8.7% | (n = 2) | 6.2% | (n = 1) | 14.3% | (n = 1) | |
| hispanic | 4.3% | (n = 1) | 0 | | 14.3% | (n = 1) | |
| Resection | | | | | | | 0.2 (pearson |
| | | | | | | | chi sq) |
| pancreatico- | 69.6% | (n = 16) | 68.8% | (n = 11) | 71.4% | (n = 5) | |
| duodenectomy | | | | | | | |
| distal | 26.1% | (n = 6) | 25% | (n = 4) | 28.6% | (n = 2) | |

| | | | | | |
|---|---|---|---|---|---|
| total | 4.3% | (n = 1) | 6.2% | (n = 1) | 0 |

The table indicates the patients' demographics of all patients included in the cohort analyzed at the University of Cincinnati. Continuous variables compared with student t-test. Categorical variables were compared with Fischer exact and Pearson chi-squared tests.

TABLE 3
Pathology data from University of Cincinnati Medicine Medical center

| | SMPD1 Low | | SMPD1 High | |
|---|---|---|---|---|
| | (n = 16) | | (n = 7) | p-value |
| | 69.6% | | 30.4% | |
| Tumor Grade | | | | 0.17 (pearson chi sq) |
| 1 - well differentiated | 6.2% | (n = 1) | 0 | |
| 2 - moderately differentiated | 75% | (n = 12) | 42.9% (n = 3) | |
| 3 - poorly differentiated | 19.8% | (n = 3) | 57.1% (n = 4) | |
| Lymph Node Involvement | 93.8% | (n = 15) | 85.7% (n = 6) | 0.53 (fisher exact) |
| Perineural Invasion | 100% | (n = 16) | 85.7% (n = 6) | 0.30 (fisher exact) |
| Lymphovascular Invasion | 62.5% | (n = 10) | 42.9% (n = 3) | 0.65 (fisher exact) |
| Stage | | | | 0.43 (pearson chi sq |
| I | 6.3% | (n = 1) | 0 | |
| IIA | 0% | (n = 0) | 14.3% (n = 1) | |
| IIB | 62.5% | (n = 10) | 57.1% (n = 4) | |
| III | 31.2% | (n = 5) | 28.6% (n = 2) | |
| Adjuvant Therapy | | | | P = 0.78 (pearson chi sq) |
| Gemcitabine-based | 56.2% | (n = 9) | 71.4% (n = 5) | |
| FOLFIRINOX | 18.8% | (n = 3) | 14.3% (n = 1) | |
| none | 25% | (n = 4) | 14.3% (n = 1) | |

The table gives the pathology characteristics of the surgery-first PDAC patients analyzed in the present study. Categorical variables were compared with Fischer exact and Pearson chi-squared tests.

TABLE 4
Human Protein Atlas Pancreatic Ductal Adenocarcinoma Cohort

| | SMPD1 Low | | SMPD1 High | | |
|---|---|---|---|---|---|
| | (n = 130) | | (n = 42) | | p-value |
| | 75.6% | | 24.4% | | |
| Age (mean, SEM) | 65.2 +/− 1.0 | | 63.0 +/− 1.7 | | 0.25 (student t-test) |
| Sex | | | | | 0.7 (Fischer Exact) |
| male | 56.2% | (n = 73) | 52.4% | (n = 22) | |
| female | 43.8% | (n = 57) | 47.6% | (n = 20) | |
| Race | | | | | 0.5 (pearson chi sq) |
| white | 88.5% | (n = 115) | 85.7% | (n = 36) | |
| black | 2.3% | (n = 3) | 7.1% | (n = 3) | |
| asian | 6.9% | (n = 9) | 4.8% | (n = 2) | |
| other, unknown | 2.3% | (n = 3) | 2.4% | (n = 1) | |
| Tumor Stage | | | | | 0.001 (pearson chi sq) |
| I | 6.2% | (n = 8) | 31.0% | (n = 13) | |
| IIA | 20% | (n = 26) | 4.8% | (n = 2) | |
| IIB | 72.3% | (n = 94) | 54.8% | (n = 23) | |
| III | 1.5% | (n = 2) | 2.4% | (n = 1) | |

| | | | |
|---|---|---|---|
| unknown | 0 | 7.1% | (n = 3) |

Patient and staging data were obtained and analyzed from the Human Protein Atlas (preoteinatlas.org). Continuous variables compared with student t-test. Categorical variables were compared with Fischer exact and Pearson chi-squared tests.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A method for predicting the survival time of an individual with pancreatic cancer comprising the steps of:
a. obtaining a tumor sample from the individual;
b. measuring acid sphingomyelinase expression in the tumor sample;
c. categorizing the level of acid sphingomyelinase expression as high expression or low expression; wherein high expression is determined by a moderate to strong level of acid sphingomyelinase shown in the tumor sample, and low expression is determined by a low to non-detectable level of acid sphingomyelinase shown in the tumor sample; and
d. using the level of acid sphingomyelinase expression to predict survival time of the individual.

2. The method as described in claim 1, wherein a high level of acid sphingomyelinase expression indicates an increased likelihood of long-term survival and an improved prognosis for the individual.

3. The method as described in claim 1, wherein a low level of acid sphingomyelinase expression indicates a decreased likelihood of long-term survival and a poor prognosis for the individual.

4. A method of treating pancreatic adenocarcinoma in an individual in need thereof comprising administering to the individual a therapeutically effective amount of a therapeutic agent comprising recombinant acid sphingomyelinase.

5. The method of claim 4 wherein the therapeutic agent further comprises one or more compositions selected from the group consisting of modified enzymes, fusion proteins and constitutively active mutants.

6. The method of claim 5 wherein the therapeutic agent further comprises a pharmaceutically acceptable excipient.

7. The method of claim 4 wherein the therapeutic agent is administered as one or more doses.

8. The method of claim 4 wherein the therapeutic agent is administered parenterally.

9. The method of claim 4 wherein the therapeutic agent is administered intravenously.

10. The method of claim 4 wherein the therapeutic agent is administered in a continuous infusion.

11. A method for predicting the survival time of an individual with pancreatic cancer and treating pancreatic cancer, comprising the steps of:
a. obtaining a tumor sample from the individual;
b. measuring acid sphingomyelinase expression in the tumor sample;
c. categorizing the level of acid sphingomyelinase expression as high expression or low expression; wherein high expression is determined by a moderate to strong level of acid sphingomyelinase shown in the tumor sample, and low expression is determined by a low to non-detectable level of acid sphingomyelinase shown in the tumor sample;
d. determining a decreased likelihood of long-term survival and a poor prognosis for an individual with a low level of acid sphingomyelinase expression; and
e. administering a therapeutically effective amount of a therapeutic agent comprising recombinant acid sphingomyelinase to an individual with a low level of acid sphingomyelinase expression.

12. The method of claim 11 wherein the therapeutic agent further comprises one or more compositions selected from the group consisting of modified enzymes, fusion proteins and constitutively active mutants.

13. The method of claim 11 wherein the therapeutic agent further comprises a pharmaceutically acceptable excipient.

14. The method of claim 11 wherein the therapeutic agent is administered as one or more doses.

15. The method of claim 11 wherein the therapeutic agent is administered parenterally.

16. The method of claim 11 wherein the therapeutic agent is administered intravenously.

17. The method of claim 11 wherein the therapeutic agent is administered in a continuous infusion.
